(19) United States
(12) Patent Application Publication
(10) Pub. No.: US 2023/0284936 A1
(43) Pub. Date: Sep. 14, 2023

(54) KITS AND METHODS FOR MEASURING INSULIN SENSATIVITY

(71) Applicant: Board of Supervisors of Louisiana State University and Agricultural and Mechanical College, Baton Rouge, LA (US)

(72) Inventor: Eric RAVUSSIN, Baton Rouge, LA (US)

(21) Appl. No.: 18/015,469

(22) PCT Filed: Jul. 12, 2021
(86) PCT No.: PCT/US2021/041284

§ 371 Date: Jan. 10, 2023

Related U.S. Application Data
(60) Provisional application No. 63/050,350, filed on Jul. 10, 2020.

(51) Int. Cl. A61B 5/145

(52) U.S. Cl. CPC A61B 5/14532; A61B 5/14507; A61B 5/14546

(57) ABSTRACT

This invention is directed to towards methods of identifying a subject with insulin resistance. For example, this invention is drawn to adjusting C-peptide urinary excretion rate for average glucose concentration to identify a subject with insulin resistance.

This application is an International Application which claims priority from U.S. provisional patent application No. 63/050,353 filed Jul. 10, 2020, the entire contents of which is incorporated herein by reference.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### FIELD OF THE INVENTION

This invention is directed towards methods of identifying a subject with insulin resistance.

### BACKGROUND OF THE INVENTION

Insulin resistance, the lack of insulin sensitivity (IS), is characterized by a decreased insulin action, which in turn leads to increased levels of insulin (hyperinsulinemia). Insulin resistance is a risk factor for multiple pathological conditions, such as type 2 diabetes mellitus and cardiovascular disease. Moreover, insulin resistance is associated with less weight loss and higher spontaneous weight gain, and some diets work better in people with low levels of insulin resistance.

### SUMMARY OF THE INVENTION

The invention provides a method of identifying a subject with insulin resistance.

In embodiments, the method comprises determining the subject's blood glucose concentration, determining the subject's blood or urinary C-peptide level calculating the ratio of the subject's blood glucose concentration to the blood or urinary C-peptide concentration, and identifying a subject with insulin resistance if this ratio is high; or identifying a subject with normal insulin sensitivity if this ratio is low, and treating the subject identified as having insulin resistance. For example, treating the subject comprises administering to the subject one or more insulin sensitizers.

In embodiments, blood glucose concentration can be measured continuously or periodically.

In embodiments, blood glucose concentration can be measured over a period of time. For example, the period of time comprises about 24 hours.

In embodiments, blood or urinary C-peptide level can be measured over a period of time. For example, the period of time comprises about 24 hours.

In embodiments, the blood glucose concentration and the urinary C-peptide level can be measured simultaneously or sequentially.

In embodiments, the subject's blood glucose concentration and/or blood C-peptide level can be measured using a transcutaneous monitor or dipstick. For example, the subject's blood glucose concentration can be measured using a transcutaneous glucose monitor (CGM).

Aspects of the invention are also drawn towards a method of identifying a subject with insulin resistance.

In embodiments, the method comprises determining the subject's blood or urinary C-peptide level, normalizing the subject's blood or urinary C-peptide level for the subject's blood glucose concentration, identifying the subject as having insulin resistance if the subject's normalized blood or urinary C-peptide level is above a threshold, and treating the subject.

In embodiments, the subject is identified as not having insulin resistance if the normalized urinary C-peptide level is below a threshold.

In embodiments, treating the subject comprises administering to the subject one or more insulin sensitizers.

In embodiments, blood glucose concentration can be measured continuously or periodically.

In embodiments, blood glucose concentration can be measured over a period of time. For example, the period of time can comprise about 24 hours.

In embodiments, blood or urinary C-peptide level can be measured over a period of time. For example, the period of time can be about 24 hours.

In embodiments, blood glucose concentration and the blood or urinary C-peptide level can be measured simultaneously or sequentially.

In embodiments, the subject's blood glucose concentration and/or blood C-peptide level can be measured using a transcutaneous monitor or dipstick. For example, the subject's blood glucose concentration can be measured using a transcutaneous glucose monitor (CGM).

Still further, aspects of the invention are drawn to a method of identifying a subject with or at risk of a disease characterized by insulin resistance.

In embodiments, the method comprises determining the subject's blood glucose concentration, determining the subject's blood or urinary C-peptide level, calculating a ratio of the subject's blood glucose concentration to the subject's blood or urinary C-peptide level, thereby identifying a subject with or at risk of a disease characterized by insulin resistance if the ratio is high, and treating the subject.

In embodiments, the disease comprises diabetes or cardiovascular disease. For example, diabetes comprises type 2 diabetes mellitus.

In embodiments, the subject can be identified as not having or not at risk of a disease characterized by insulin resistance if the ratio is low.

In embodiments, treating the subject can comprise administering to the subject one or more insulin sensitizers.

In embodiments, blood glucose concentration can be measured continuously or periodically.

In embodiments, blood glucose concentration can be measured over a period of time. For example, the period of time can comprise about 24 hours.

In embodiments, blood or urinary C-peptide level can be measured over a period of time. For example, the period of time can be about 24 hours.

In embodiments, blood glucose concentration and the blood or urinary C-peptide level can be measured simultaneously or sequentially.

In embodiments, the subject's blood glucose concentration and/or blood C-peptide level can be measured using a transcutaneous monitor or dipstick. For example, the subject's blood glucose concentration can be measured using a transcutaneous glucose monitor (CGM).

Still further, aspects of the invention are drawn to a method of identifying a subject with or at risk of a disease characterized by insulin resistance.

In embodiments, the method comprises determining the subject's blood or urinary C-peptide level, normalizing the subject's blood or urinary C-peptide level for the subject's blood glucose concentration, identifying the subject as having insulin resistance if the subject's normalized blood or urinary C-peptide level is above a threshold, and treating the subject.

In embodiments, the disease comprises diabetes or cardiovascular disease. For example, diabetes comprises type 2 diabetes mellitus.

In embodiments, the subject can be identified as not having or not at risk of a disease characterized by insulin resistance if the normalized blood or urinary C-peptide level is below a threshold.

In embodiments, treating the subject comprises administering to the subject one or more insulin sensitizers.

In embodiments, blood glucose concentration can be measured continuously or periodically.

In embodiments, blood glucose concentration can be measured over a period of time. For example, the period of time can be about 24 hours.

In embodiments, blood or urinary C-peptide level can be measured over a period of time. For example, the period of time is about 24 hours.

In embodiments, the subject's blood glucose concentration and/or blood C-peptide level can be measured using a transcutaneous monitor or dipstick. For example, the subject's blood glucose concentration can be measured using a transcutaneous glucose monitor (CGM).

Also, aspects of the invention are drawn towards a method of determining a subject's C-peptide level.

In embodiments, the method comprises obtaining a blood and/or urine sample from a subject, measuring the blood or urinary C-peptide level, measuring blood glucose concentration in the subject, and normalizing the blood or urinary C-peptide level for the subject's blood glucose concentration, and thereby determining the subject's C-peptide level.

In embodiments, the subject's C-peptide level is an indicator of the subject's resistance to insulin.

In embodiments, the subject's C-peptide level is an indicator of the subject having or at risk of having a disease characterized by insulin resistance.

In embodiments, the disease comprises diabetes or cardiovascular disease. For example, diabetes comprises type 2 diabetes mellitus.

Further, aspects of the invention are drawn to a kit.

In embodiments, the kit comprises one or more components and reagents to measure blood glucose concentration in a subject, blood C-peptide level in a subject, or urinary C-peptide in a subject.

Other objects and advantages of this invention will become readily apparent from the ensuing description.

### BRIEF DESCRIPTION OF THE FIGURES

FIG. 1 shows an exemplary, non-limiting, experimental protocol for CGM: continuous glucose monitoring device.

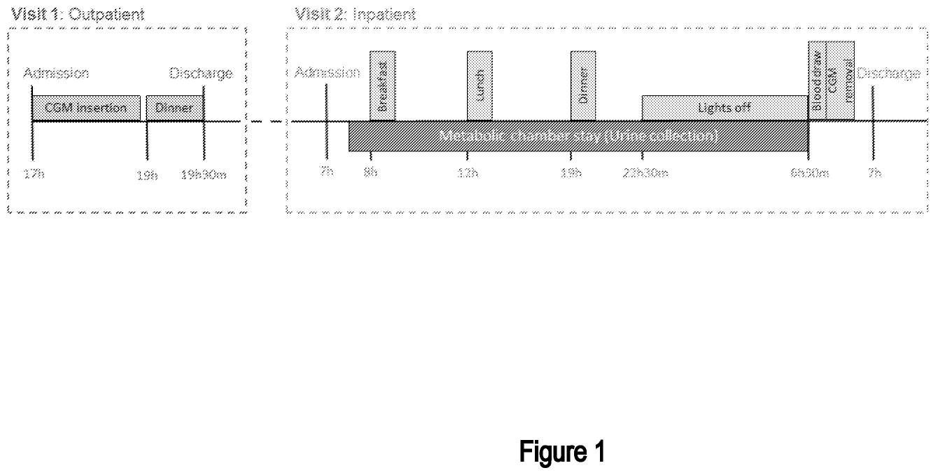

FIG. 2 shows an exemplary protocol for a euglycemic-hyperinsulinemic claim. This method is expensive, presents high burden for participants, and assesses insulin sensitivity under non-physiological conditions.

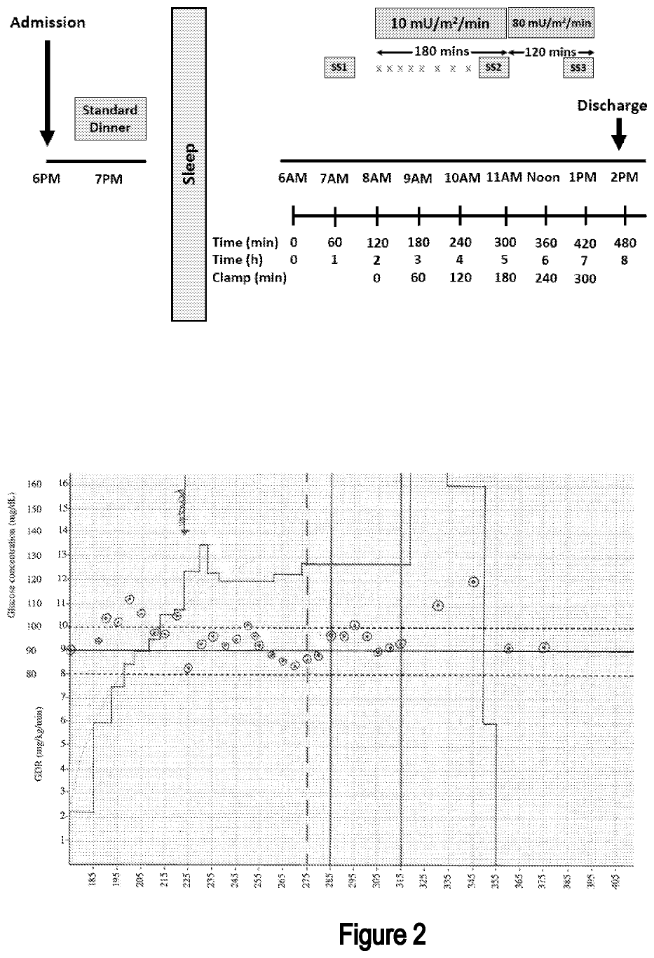

FIG. 3 a non-limiting exemplary graph of insulin-stimulated glucose disposal rate (mg/kg EMBS/min) vs. 24 h Urinary C-peptide excretion (n/kcal).

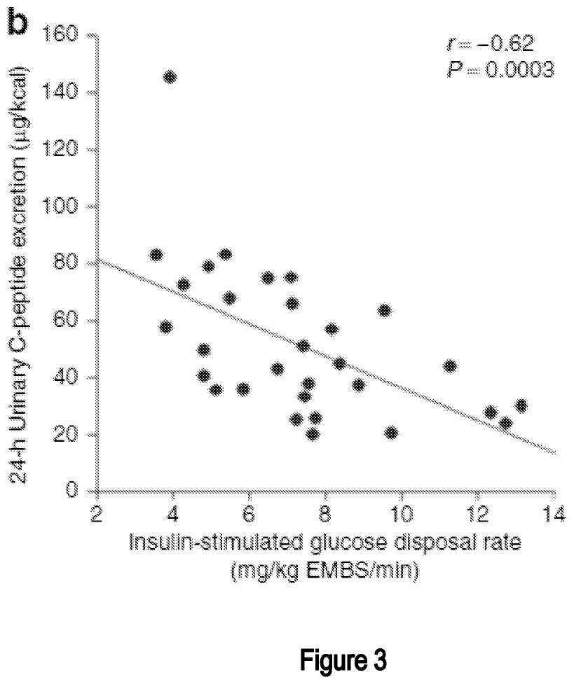

FIG. 4 shows examples of images of a glucose monitor and urine collection.

### DETAILED DESCRIPTION OF THE INVENTION

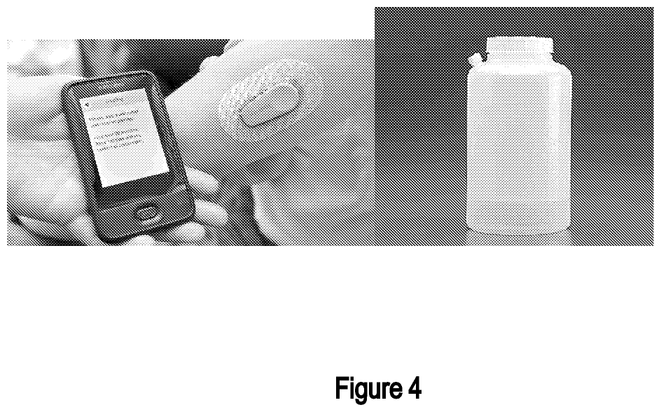

The 24-hour C-peptide urinary excretion rate, adjusted for energy intake, is better correlated with insulin sensitivity than other indirect methods. (Galgani et al; Obesity 18, 1852-57, 2010) (FIG. 4). However, the circulating glucose concentration is the major stimulus for insulin secretion and it varies in response to identical food intake from one person to another. As such, adjusting C-peptide urinary excretion rate for average glucose concentration instead of energy/nutrient intake can improve the prognosis of insulin sensitivity.

Detailed descriptions of one or more preferred embodiments are provided herein. It is to be understood, however, that the invention can be embodied in various forms. Therefore, specific details disclosed herein are not to be interpreted as limiting, but rather as a basis for the claims and as a representative basis for teaching one skilled in the art to employ the invention in any appropriate manner.

The singular forms “a”, “an” and “the” include plural reference unless the context clearly dictates otherwise. The use of the word “a” or “an” when used in conjunction with the term “comprising” in the claims and/or the specification can mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

As used herein the term “about” is used herein to mean approximately, roughly, around, or in the region of. When the term “about” is used in conjunction with a numerical range, it modifies that range by extending the boundaries above and below the numerical values set forth. The term “about” is used herein to modify a numerical value above and below the stated value by a variance of 20 percent up or down (higher or lower).

Aspects of the invention are directed towards methods of identifying a subject with insulin resistance. The term “insulin resistance” can refer to a state in which a normal amount of insulin produces a subnormal biologic response relative to the biological response in a subject that does not have insulin resistance.

Insulin resistance can be characterized by a decreased insulin action, which in turn leads to increased levels of insulin (hyperinsulinemia). The term “hyperinsulinemia” can refer to a state in an individual in which the level of insulin in the blood is higher than normal.

The term “insulin” can refer to a peptide that is secreted by the pancreas in response to the elevated blood glucose levels in the body to take up glucose into the liver, muscle, or adipose tissue, turns it into glycogen, and to stop the use of fat as an energy source, and thus functions to control blood glucose. This peptide includes native insulin, basal insulin, and the agonists, precursors, derivatives, fragments, and variants thereof.

As used herein, “native insulin” refers to a hormone that is secreted by pancreas to promote glucose absorption but inhibit fat breakdown in the cells and thus functions to control the blood glucose level. Insulin is generated by processing its precursor, proinsulin, which does not have a function of regulating blood glucose level. The amino acid sequences of insulin are as follows:

Alpha chain
(SEQ ID NO. 1)
Gly-Ile-Val-Glu-Gln-Cys-Cys-Thr-Ser-Ile-
Cys-Ser-Leu-Tyr-Gln-Leu-Glu-Asn-Tyr-Cys-
Asn
Beta chain:
(SEQ ID NO. 2)
Phe-Val-Asn-Gln-His-Leu-Cys-Gly-Ser-His-
Leu-Val-Glu-Ala-Leu-Tyr-Leu-Val-Cys-Gly-
Glu-Arg-Gly-Phe-Phe-Tyr-Thr-Pro-Lys-Thr

“Basal insulin” refers to insulin that regulates glucose levels between measle and is released 24 hours a day, whether or not a person eats.

Proinsulin-like components (proinsulin and its intermediate forms, PLC) and C-peptide have been identified as secretory products of the pancreatic beta cells in addition to insulin.

The term “C-peptide” (C₁₂₉H₂₁₁N₃₅O₄₈, 31 amino acid polypeptide) can refer to the connecting peptide which connects the A and B chains of the insulin protein hormone involved in the regulation of blood sugar levels. Insulin is produced in the liver as: its precursor proinsulin, consisting of the B and A chains of insulin linked together via a connecting C-peptide (hereinafter this C-peptide derived from the proinsulin molecule is referred to as “insulin C-peptide”). The term c-peptide also encompasses species variants, homologues, allelic forms, mutant forms, and equivalents thereof, including conservative substitutions, additions, deletions therein not adversely affecting the structure of function. The amino acid sequence of C-peptide is as follows:

(SEQ ID NO: 3)
Glu-Ala-Glu-Asp-Leu-Gln-Val-Gly-Gln-Val-
Glu-Leu-Gly-Gly-Gly-Pro-Gly-Ala-Gly-Ser-
Leu-Gln-Pro-Leu-Ala-Leu-Glu-Gly-Ser-Leu-
Gln

See, for example, Ko, Arthur S C, et al. “The amino acid sequence of the C-peptide of human proinsulin.” European journal of biochemistry 20.2 (1971): 190-199.

A normal range for C-peptide level can be between 0.5 and 10 ng/mL. For example, the normal range can be between 0.9 and 7.1 ng/mL. For example, a normal, blood C-peptide level can be between 0.5 and 2.0 nanograms per milliliter (ng/mL), or 0.17 to 0.83 nanomoles per liter (nmol/L).

Normal, 24-hour urine C-peptide excretion rate has been reported as 56.7±22.0 ug/day in study participants with normal glucose tolerance test while being 115.4±40.2 ug/day in patients with type 2 diabetes (p<0.0001). See, for example, Chung, Young Hwan, et al. “High 24-Hour Urinary C-Peptide Excretion in Non-Insulin Dependent Diabetes Mellitus.” The Korean journal of internal medicine 1.2 (1986): 172.

A low level (or no C-peptide) indicates that a subject's pancreas is producing little or no insulin. A low C-peptide level can be normal if a subject has not eaten recently (in which case the subject's blood sugar and insulin levels can be naturally be low then as well). A low C-peptide level is abnormal if a subject's blood sugar is high, as the subject's body can be making insulin at that time. Subject's with type 2 diabetes, obesity, or insulin resistance can have a high C-peptide level, which means the subject's body is producing a lot of insulin to keep their blood sugar normal.

A subject's C-peptide level, adjusted for energy intake, can be better correlated with insulin sensitivity. However, the circulating glucose concentration is the major stimulus for insulin secretion and it can vary in response to identical food intake (i.e., energy intake) from one person to another. As such, adjusting C-peptide level for average glucose concentration instead of energy/nutrient intake can improve the prognosis of insulin sensitivity, for example, since different dietary carbohydrates have different impact on blood glucose concentration.

The term “subject” can refer to any organism to which aspects of the invention can be performed, e.g., for experimental, diagnostic, prophylactic, and/or therapeutic purposes. Example of subjects include be mammals, such as primates, for example humans. For veterinary applications, a wide variety of subjects will be suitable, e.g., livestock such as cattle, sheep, goats, cows, swine, and the like; poultry such as chickens, ducks, geese, turkeys, and the like; and domesticated animals such as pets, for example dogs and cats. For diagnostic or research applications, a wide variety of mammals will be suitable subjects, including rodents (e.g., mice, rats, hamsters), rabbits, primates, and swine such as inbred pigs and the like. The term “living subject” can refer to a subject noted herein or another organism that is alive. The term “living subject” can refer to the entire subject or organism and not just a part excised (e.g., a liver or other organ) from the living subject.

In embodiments, the method comprises determining a subject's glucose concentration and determining the subject's C-peptide level. From these two values, one can calculate a ratio of the subject's glucose concentration to the subject's C-peptide level, as using the ratio to identify a subject with insulin resistance. In embodiments, the ratio can be about greater than 1000:1, about 1000:1, about 900:1, about 800:1, about 700:1, about 600:1, about 500:1, about 400:1, about 300:1, about 200:1, about 100:1, about 90:1, about 80:1, about 70:1, about 60:1, about 50:1, about 40:1, about 30:1, about 20:1, about 10:1, about 5:1, about 1:1, less than about 1:1, about 1:5, about 1:10, about 1:20, about 1:30, about 1:40, about 1:50, about 1:60, about 1:70, about 1:80, about 1:90, about 1:100, about 1:200, about 1:300, about 1:400, about 1:500, about 1:600, about 1:700, about 1:800, about 1:900, about 1:1000, or greater than about 1:1000.

For example, the subject can be identified as having insulin resistance if the ratio is high. For example, the subject can be identified as having insulin resistance if the ratio is above a threshold. For example, the subject can be identified as having insulin resistance if the ratio is high when compared to a control sample.

In embodiments, the subject's C-peptide level and/or glucose concentration can be measured in a subject in a fasting (e.g., deprived of glucose) or fed (e.g., stimulated with glucose) state. By way of example, a subject in a fasting state can abstain from food for about 30 minutes, about 1 hour, about 2 hours, about 4 hours, about 6 hours, about 8 hours, about 12 hours, about 18 hours, about 20 hours, or about 24 hours prior to analysis of the subject's C-peptide level and/or blood glucose concentration. In another example, a subject in a fed state can consume food within 12 hours, within 10 hours, within 6 hours, within 4 hours, within 3 hours, within 2 hours, within 1.5 hours, within 1 hour, within 30 minutes, within 15 minutes, or concurrent with analysis of a subject's C-peptide level and/or glucose concentration.

The term “blood glucose concentration” can refer to the glucose concentration in the subject's bloodstream. The normal blood glucose concentration (normoglycemia) is approximately 85-95 mg/dl in an overnight fasting state. This value varies up to 30 mg/dl if not diabetic. “Hyperglycemia” is a situation in which the blood glucose concentration is too high. Hyperglycemia can occur when blood glucose levels rise and exceed 180 mg/dl.

The term “urinary glucose concentration” can refer to the glucose concentrating in the subject's urine. The normal amount of glucose in urine is 0 to 0.8 mmol/L. A higher measurement can be a sign of health problems, such as diabetes.

The skilled artisan will recognize that any composition and/or method to measure a subject's glucose concentration can be utilized in aspects of the invention. Further compositions and/or methods to measure a subject's glucose concentration are known in the art. For example, the subject's blood glucose concentration can be measure using a chemically-treated, disposable “test-strip”, which is then inserted into an electronic blood glucose meter. See Pickering, Dianne, and Janet Marsden. “How to measure blood glucose.” Community eye health 27.87 (2014): 56. As another example, a colorimetric assay can be used. Other assays comprise enzymatic assays, such as those based on the glucose oxidase enzyme. As described herein, continuous glucose monitors can also be used to measure a subject's glucose concentration. Further, a dipstick test can be used to perform a urine glucose test.

In embodiments, the subject's glucose concentration and/or the subject's C-peptide level can be measured once, or can be measured continuously or periodically over a period of time. The term “period of time” can refer to the period of time necessary to achieve an effect or result. For example, the period of time can comprise about 1 hour, about 2 hours, about 3 hours, about 4 hours, about 5 hours, about 6 hours, about 12 hours, about 18 hours, about 24 hours, about 36 hours, about 48 hours, or longer than about 48 hours. In embodiments, the period of time comprises about 12 or about 24 hours.

The term “periodically” can refer to recurring or repeating events at determinable intervals. The periodic intervals can be evenly spaced or unevenly spaced time intervals.

The term “continuously” can refer to without interruption or with minimal interruption. For example, continuous measurement can refer to the fact that the measurements are repeated continuously over very small intervals of time. In embodiments, the subject's C-peptide level and/or glucose level can be measure continuously over a period of time.

For example, a subject's blood glucose concentration can be measured continuously using a continuous glucose monitor (CGM) or continuous glucose sensor. The term “continuous glucose monitor” or “continuous glucose sensor” can refer to a device that continuously or continually measures the glucose concentration of a bodily fluid (e.g., blood, plasma, interstitial fluid and the like), for example, at time intervals ranging from fractions of a second up to, for example, 1, 2, or 5 minutes, or longer. Continual or continuous glucose sensors can continually measure glucose concentration without requiring user initiation and/or interaction for each measurement.

The term “continuous glucose sensing” or “continuous glucose monitoring” can refer to the period in which monitoring of the glucose concentration of a host's bodily fluid (e.g., blood, serum, plasma, extracellular fluid, etc.) is continuously or continually performed, for example, at time intervals ranging from fractions of a second up to, for example, 1, 2, or 5 minutes, or longer. In one exemplary embodiment, the glucose concentration of a host's extracellular fluid is measured every 1, 2, 5, 10, 20, 30, 40, 50 or 60-seconds.

In embodiments, continuous glucose monitors can accurately and precisely measure blood glucose levels in a subject for a period of time, such as up to 1 hour, 2 hours, 3 hours, 4 hours, 5 hours, 6 hours, 12 hours, 1 day, 2 days, 3 days, 7 days, 14 days, 21 days, 28 days, or longer than 28 days.

Embodiments described herein can refer to the use of a glucose sensor or glucose monitor that measures a concentration of glucose or a substance indicative of the concentration or presence of the analyte. In embodiments, the glucose sensor is a continuous device, for example a subcutaneous, transdermal, transcutaneous, non-invasive, and/or intravascular (e.g., intravenous) device. In embodiments, the device can analyze a plurality of intermittent blood samples. The glucose sensor can use any method of glucose-measurement, including enzymatic, chemical, physical, electrochemical, optical, optochemical, fluorescence-based, spectrophotometric, spectroscopic (e.g., optical absorption spectroscopy, Raman spectroscopy, etc.), polarimetric, calorimetric, iontophoretic, radiometric, and the like.

The glucose sensor can use any known detection method, including invasive, minimally invasive, and non-invasive sensing techniques, to provide a data stream indicative of the concentration of the analyte in a host. The data stream can be a raw data signal that is used to provide a useful value of the analyte to a user, such as a patient or health care professional (e.g., doctor), who can be using the sensor.

In embodiments, the continuous glucose sensor can comprise a glucose sensor configured to measure glucose in the blood using one or more measurement techniques, such as enzymatic, chemical, physical, electrochemical, fluorescent, spectrophotometric, polarimetric, calorimetric, iontophoretic, radiometric, or immunochemical. In embodiments, the continuous glucose sensor can comprise any device that can measure the concentration of glucose and can use a variety of techniques to measure glucose including invasive, minimally invasive, and non-invasive sensing techniques (e.g., fluorescent monitoring), to provide a data, such as a data stream, indicative of the concentration of glucose in a host. The data stream can be raw data signal, which is converted into a calibrated and/or filtered data stream used to provide a value of glucose to a subject, such as a user, a patient, or a caretaker (e.g., a parent, a relative, a guardian, a teacher, a doctor, a nurse, or any other individual that has an interest in the wellbeing of the subject). Moreover, the continuous glucose sensor can be implanted as at least one of the following types of sensors: an implantable glucose sensor, a transcutaneous glucose sensor, implanted in a host vessel or extracorporeally, a subcutaneous sensor, a refillable subcutaneous sensor, an intravascular sensor.

As with the continuous glucose sensors described herein, embodiments can also comprise the continuous transcutaneous measurement of C-peptide in a subject's blood.

A healthy subject (e.g., a subject without autoimmune diabetes) has an endogenous C-peptide level that ranges from about 0.6 nmol/L to about 0.8 nmol/L (e.g., about 0.65 nmol/L). In contrast, a subject with autoimmune diabetes (e.g., Type 1 diabetes or LADA) has an endogenous C-peptide level ranging from undetectable to about 0.6 nmol/L (e.g., about 0.05 nmol/L).

In embodiments, the subject's C-peptide level and/or glucose level can be measured in a biological sample, such as in a subject's blood or urine. The biological sample can be a biological fluid, i.e., a bodily fluid. The bodily fluid comprises peripheral blood, sera, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, female ejaculate, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, blastocyl cavity fluid, or umbilical cord blood. In some embodiments, the biological sample comprises blood or a blood derivative, such as peripheral blood, sera, or plasma. In embodiments, the subject's glucose level is measured in blood or urine. In embodiments, the subject' C-peptide level is measured in blood or urine.

The methods described herein can involve obtaining a biological sample from the subject. As used herein, the phrase “obtaining a biological sample” can refer to any process for directly or indirectly acquiring a biological sample from a subject. For example, a biological sample can be obtained (e.g., at a point-of-care facility, e.g., a physician's office, a hospital, laboratory facility) by procuring a tissue or fluid sample (e.g., blood draw, marrow sample, spinal tap) from a subject. Alternatively, a biological sample can be obtained by receiving the biological sample (e.g., at a laboratory facility) from one or more persons who procured the sample directly from the subject. The biological sample can be, for example, a tissue (e.g., blood), cell (e.g., hematopoietic cell such as hematopoietic stem cell, leukocyte, or reticulocyte, stem cell, or plasma cell), vesicle, biomolecular aggregate or platelet from the subject.

In embodiments, the subject's urine can be collected, and analytes (such as C-peptide or glucose) can be measured. Analytes in the urine can be measure by assays and techniques known in the art, including urinary sticks (e.g., dip-sticks), lateral flow assays (such as lateral flow immunoassays). An assay can detect the presence and/or amount of more than one analyte.

In embodiments, the subject's glucose concentration and the subject's C-peptide level are measure simultaneously or sequentially. For example, the term “simultaneous measurement” can refer to the measurement of two or more analytes at substantially the same time, optionally, in the same sample. The term “sequential” or “sequentially” can refer to the measurement of the two or more analytes one after the other (i.e., not at the same time) In embodiments, the same sample can be used for the sequential measurements (such as the same blood sample or the same urine sample). In embodiments, different samples can be used for the sequential measurements (such as a blood sample to measure glucose levels, and a urine sample to measure c-peptide levels).

In embodiments, a subject is identified as having or not having insulin resistance based on a ratio of the subject's glucose concentration to the subject's C-peptide level. The term “ratio” can refer to the relative amount of one or more compounds in relation to another compound or compounds. As an example, the subject can be identified as having insulin resistance if the ratio of a subject's blood glucose concentration to the subject's urinary C-peptide level is low. As an example, the subject can be identified as not having insulin resistance (such as a subject with normal insulin sensitivity) if the ratio of a subject's blood glucose concentration to the subject's urinary C-peptide level is high.

In embodiments, a subject is identified as having or not having insulin resistance based on whether the ratio of the subject's glucose concentration to the subjects C-peptide level is above or below a threshold. The term “threshold” can refer to a value derived from a plurality of biological samples, such as donor blood samples or donor urinary samples, above which threshold is associated with an increased likelihood of having and/or developing insulin resistance.

In embodiments, the ratio of the subject's glucose concentration to the subject's C-peptide level can be compared to a control sample to determine whether or not the subject has or is at risk of developing insulin resistance. “Changed as compared to a control” sample or subject is understood as having a level of an indicator(s) to be detected at a level that is statistically different than a sample from a normal, untreated, or abnormal state control sample. Determination of statistical significance is within the ability of those skilled in the art, e.g., the number of standard deviations from the mean that constitute a positive or negative result.

Embodiments as described herein can provide a scaling of insulin sensitivity. For example, embodiments can identify a subject having:
Low insulin sensitivity (i.e. insulin resistance)
Normal insulin sensitivity
High insulin sensitivity

For example, a subject with “low insulin sensitivity” (i.e., insulin resistance) can refer to a subject whose cells do not absorb as much glucose as a normal subject, which can lead to excessively high blood sugar levels (i.e., hyperinsulinemia), and a subject with “high insulin sensitivity” can refer to a subject whose cells absorb more glucose than a normal subject.

In embodiments, the method comprises treating a subject, such as a subject identified as having insulin resistance. The term “treating” can refer to partially or completely alleviating, ameliorating, improving, relieving, delaying onset of, inhibiting progression of, reducing severity of, and/or reducing incidence of one or more symptoms, features, or clinical manifestations of a disease, disorder, and/or condition, such as a disease characterized by insulin resistance. For example, “treating” insulin resistance can refer improving the sensitivity of the subject to insulin, reducing or normalizing glucose levels. For example, “treating” insulin resistance can include lifestyle interventions (such as, diet and physical activity) to manage body weight, pharmacological interventions for weight loss, pharmacological treatment with insulin sensitizers, bariatric surgery, or a combination thereof. Treatment can be administered to a subject who does not exhibit signs of a disease, disorder, and/or condition (e.g., prior to an identifiable disease, disorder, and/or condition), and/or to a subject who exhibits only early signs of a disease, disorder, and/or condition for the purpose of decreasing the risk of developing pathology associated with the disease, disorder, and/or condition.

In embodiments, treatment comprises administering to the subject one or more therapeutic agents. The term “therapeutic agent” can refer to any chemical moiety that is a biologically, physiologically, or pharmacologically active substance that acts locally or systemically in a subject. The term also can refer to any substance intended for use in the diagnosis, cure, mitigation, treatment or prevention of disease or in the enhancement of desirable physical or mental development and/or conditions in an animal or human. For example, the therapeutic agent can be an insulin sensitizer.

The term “insulin sensitizer” can refer to an agent that improves the sensitivity of cells to the metabolic effects of insulin when administered to a patient (e.g., patient with insulin resistance, diabetes). Examples thereof include metformin and peroxisome proliferator-activated receptor (PPAR) γ agonist (e.g., thiazolidinedione compounds such as pioglitazone, rosiglitazone, troglitazone, ciglitazone and the like, nonthiazolidinedione compounds such as GI-262570, GW-1929, JTT-501, YM-440 and the like, etc.), PPARα antagonist (e.g., bisphenol A diglycidyl ether, LG-100641 etc.), PPARα agonist (fibrate compounds such as clofibrate, bezafibrate, clinofibrate and the like, nonfibrate compounds etc.), PPARα/γ agonist (e.g., KRP-297 etc.), retinoid X receptor agonist (e.g., LG-100268 etc.), retinoid X receptor antagonist (e.g., HX531 etc.), protein tyrosine phosphatase-1B inhibitor (e.g., PTP-112 etc.).

In embodiments, treatment can comprise one or more weight loss interventions (e.g., lifestyle, pharmacological, and/or surgical interventions).

Aspects of the invention are further drawn to methods, for example to identify a subject with insulin resistance, comprising determining the subject's urinary C-peptide level, normalizing the subject's urinary C-peptide level for the subject's blood glucose concentration, and identifying the subject as having insulin resistance if the subject's normalized urinary C-peptide level is high (i.e., above a threshold), or having normal insulin sensitivity if the subject's normalized urinary C-peptide level is low (i.e., below a threshold).

In embodiments, the subject's normalized urinary C-peptide level can provide a scaling of insulin sensitivity. The term “insulin sensitivity” can refer to the ability of a cell to regulate glucose uptake and utilization in response to the action of insulin. Insulin sensitivity can range from:
Low insulin sensitivity (i.e. insulin resistance)
Normal insulin sensitivity
High insulin sensitivity

Aspects of the invention are also drawn towards methods of identifying a subject with or at risk of a disease characterized by insulin resistance.

An “insulin resistance disorder” can refer to a disease or condition that is caused by or contributed to by insulin resistance. Examples include: diabetes, obesity, metabolic syndrome, insulin-resistance syndromes, syndrome X, insulin resistance, high blood pressure, hypertension, high blood cholesterol, dyslipidemia, hyperlipidemia, dyslipidemia, atherosclerotic disease including stroke, coronary artery disease or myocardial infarction, hyperglycemia, hyperinsulinemia and/or hyperproinsulinemia, impaired glucose tolerance, delayed insulin release, diabetic complications, including coronary heart disease, angina pectoris, congestive heart failure, stroke, cognitive functions in dementia, retinopathy, peripheral neuropathy, nephropathy, glomerulonephritis, glomerulosclerosis, nephrotic syndrome, hypertensive nephrosclerosis some types of cancer (such as endometrial, breast, prostate, and colon), complications of pregnancy, poor female reproductive health (such as menstrual irregularities, infertility, irregular ovulation, polycystic ovarian syndrome (PCOS)), lipodystrophy, cholesterol related disorders, such as gallstones, cholescystitis and cholelithiasis, gout, obstructive sleep apnea and respiratory problems, osteoarthritis, and prevention and treatment of bone loss, e.g. osteoporosis.

In embodiments, the disease can be a metabolic disease. The term “metabolic disease” or “metabolic disorder” can refer to any disease or disorder that disrupts normal metabolism, including any disease that disrupts or dysregulates biochemical reactions that function to convert food into energy, process or transport amino acids, proteins, carbohydrates (e.g., sugars, starches), or lipids (e.g., fatty acids), etc. In embodiments, a metabolic disease results in the abnormal processing or regulation of sugars, lipids, cholesterol, and/or the metabolism of drugs (e.g., by the liver). Non-limiting examples of metabolic diseases include obesity, insulin resistance, type 2 diabetes, hyperlipidemia, non-alcoholic fatty liver disease (NAFLD), and non-alcoholic steatohepatitis (NASH), as well as the sequelae of such diseases.

For example, the term “diabetes” can refer to high blood sugar or ketoacidosis, as well as chronic metabolic abnormalities arising from a prolonged high blood sugar status or a decrease in glucose tolerance. “Diabetes” encompasses both the type I and type II (Non Insulin Dependent Diabetes Mellitus or NIDDM) forms of the disease. The risk factors for diabetes include the following factors: waistline of more than 40 inches for men or 35 inches for women, blood pressure of 130/85 mmHg or higher, triglycerides above 150 mg/dl, fasting blood glucose greater than 100 mg/dl or high-density lipoprotein of less than 40 mg/dl in men or 50 mg/dl in women. For example, diabetes can refer to type 2 diabetes mellitus.

In embodiments, the disease can be a cardiovascular disease. The term “cardiovascular disease” can refer to diseases and disorders of the heart and circulatory system. Exemplary cardiovascular diseases, including cholesterol- or lipid-related disorders, include, but are not limited to, acute coronary syndrome, angina, arteriosclerosis, atherosclerosis, carotid atherosclerosis, cerebrovascular disease, cerebral infarction, congestive heart failure, congenital heart disease, coronary heart disease, coronary artery disease, coronary plaque stabilization, dyslipidemias, dyslipoproteinemias, endothelium dysfunctions, familial hypercholeasterolemia, familial combined hyperlipidemia, hypoalphalipoproteinemia, hypertriglyceridemia, hyperbetalipoproteinemia, hypercholesterolemia, hypertension, hyperlipidemia, intermittent claudication, ischemia, ischemia reperfusion injury, ischemic heart diseases, cardiac ischemia, metabolic syndrome, multi-infarct dementia, myocardial infarction, obesity, peripheral vascular disease, reperfusion injury, restenosis, renal artery atherosclerosis, rheumatic heart disease, stroke, thrombotic disorder, transitory ischemic attacks, and lipoprotein abnormalities associated with Alzheimer's disease, obesity, diabetes mellitus, syndrome X, impotence, multiple sclerosis, Parkinson's diseases and an inflammatory diseases.

Further aspects of the invention are drawn towards methods of determining a subject's C-peptide level.

In embodiments, the method comprises obtaining a sample from a subject and measuring the C-peptide level, measuring glucose concentration in the subject, and normalizing the C-peptide level for the subject's glucose concentration. Thereby, the subject's normalized C-peptide level is determined.

In embodiments, the subject's C-peptide level and/or glucose concentration can be measured in vivo, such as via a transcutaneous sensor.

In embodiments, the C-peptide level is an indicator of the subject's resistance to insulin, and/or the subject having or at risk of having a disease characterized by insulin resistance. The term “indicator” can refer to the result of a test or examination that can provide feedback as to whether a subject has or is at risk of having a disease.

Further aspects of the invention are drawn towards a kit comprising components and/or reagents that can measure blood glucose concentration and urinary C-peptide levels in a subject.

The term “kit” can refer to a product (i.e., a kit of parts) comprising one package or one or more separate packages, and including informational material. In embodiments, the kit can comprise one or more components and reagents that can measure glucose concentration in a subject, and/or one or more components and reagents that can measure C-peptide levels in a subject.

In embodiments, the components can comprise one or more disposable articles for measuring glucose concentration and/or C-peptide levels. The term “disposable article” can refer to a single or limited use article that is made from relatively inexpensive materials that make the article cost effective to fabricate. For example, the disposable article can be a swab, spoon, dipstick, filter paper, or test-strip.

In embodiments, the components can comprise a medical device. The term “medical device” can refer any instrument, apparatus, implant, in vitro reagent or similar or related article that is used to diagnose, prevent, or treat a disease or other condition, and does not achieve its purpose through pharmacological action within or on the body. For example, the medical device can be a sensor, such as a glucose sensor or a sensor to measure C-peptide levels.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1—Method to Measure Insulin Sensitivity in Humans

An aspect of this invention comprises a method to determine insulin sensitivity in physiological conditions (unlike the hyperinsulimic clamp which is the standard) by combining 24-hour interstitial glucose contraction measured by CGM (Continuous Glucose Monitoring) and 24-hour urinary c-peptide excretion rate. The rate of 24-hour C-peptide urinary excretion normalized for the average 24-hour glucose concentration (or an algorithm of the profile) is a physiological measure of whole body insulin resistance (the higher the ratio, the higher the insulin resistance).

Without wishing to be bound by theory, embodiments of the invention can comprise a kit or apparatus which combines measures of interstitial glucose, urinary volume and C-peptide concentration.

Embodiments described herein provide advantages to measuring insulin sensitivity in comparison to invasive methods such as the glucose clamp, the frequently sampled intravenous glucose tolerance or the oral glucose tolerance test.

### Example 2—Development of a Method to Measure Insulin Sensitivity in Humans: A Study “CGM-PEPTIDE”

Background

Insulin resistance, the lack of insulin sensitivity (IS), is characterized by a decreased insulin action, which in turn leads to increased levels of insulin (hyperinsulinemia)¹. The term “hyperinsulinemia” can refer to a state in an individual in which the level of insulin in the blood is higher than normal.

Insulin resistance is a risk factor for multiple pathological conditions, such as type 2 diabetes mellitus and cardiovascular disease². Moreover, insulin resistance is associated with less weight loss and higher spontaneous weight gain³⁻⁶, and some diets work better in people with low levels of insulin resistance⁷⁻¹⁰. The euglycemic-hyperinsulinemic clamp is considered the standard for the assessment of IS in humans. However, this method is expensive, presents high burden for participants and assess IS under non-physiological conditions (receiving exogenous glucose infusion)¹¹. Alternative IS markers are based on the plasma glucose/insulin circulating levels in fasting or post-prandial conditions^{12,13}.

Galgani et al.¹¹ developed a simpler measure of IS based on the assessment of 24-hour C-peptide urinary excretion under controlled feeding conditions. C-peptide is secreted in equimolar quantities with insulin by pancreas and, although it is not completely excreted in urine, its urinary excretion rate provides an indirect, non-invasive, simple marker of insulin secretion. Indeed, Galgani et al.¹¹ showed that the 24-hour C-peptide urinary excretion rate, adjusted for energy intake, provides a stronger correlation (r=0.62) with IS determined by the euglycemic-hyperinsulinemic clamp than other simple (e.g. HOMA-IR, Matsuda index) or complex (deuterated-glucose disposal test) methods.

However, the circulating glucose concentration is the major stimulus for insulin secretion, and glucose concentration in response to identical food intake has large inter-individual variability. Therefore, without wishing to be bound by theory, adjusting C-peptide urinary levels for average glucose concentration instead of energy/nutrient intake can improve the prognosis of IS compared to that reported by Galgani et al.¹¹. Therefore, without wishing to be bound by theory, the new methodology to continuously measure glucose concentration (continuous glucose monitoring=CGM) will provide a better normalizer of C-peptide production for IS determination than energy/nutrient intake¹⁴.

Study

This study will collect data to validate the combination of 24 h C-peptide urinary excretion rate and CGM to assess IS in humans. Specifically, this study will validate that the ratio between 24 h C-peptide urinary excretion rate and average 24 h circulating glucose represent a good correlation of what is measured by the standard, i.e. M (glucose disposal rate) from a euglycemic-hyperinsulinemic clamp. We will perform this study in twelve study participants with different levels of insulin sensitivity.

Without wishing to be bound by theory:
The 24 h C-peptide urinary levels/24 h circulating glucose ratio is associated to the previously measured M-value at the high-dose (≥80 mIU/m²/min) of a euglycemic-hyperinsulinemic clamp.
The effect size (r) of the relationship between 24 h C-peptide urinary levels/24 h circulating glucose ratio and the M-value will be larger than the effect size (r) of the relationship between the 24 h C-peptide urinary levels/energy intake ratio or HOMA-IR with the M-value.

Non-Limiting Exemplary Research Design

Without wishing to be bound by theory, we will conduct a cross-sectional, observational, study in twelve subjects, whose insulin sensitivity has been previously measured by a high-dose euglycemic-hyperinsulinemic clamp. Participants will be admitted to the research clinic for a 24-hour stay in a metabolic chamber. During the chamber stay, all urine excreted will be collected to assess C-peptide urinary excretion rate and interstitial glucose will be measured by a continuous glucose monitor (CGM). Participants will consume a eucaloric diet (50% carbohydrates, 30% fat and 20% protein).

Non-Limiting Exemplary Study Population

Subject Population

Up to twelve healthy men or women will be enrolled. If eligible and enrolled, individuals will complete a screening visit, one short visit in the afternoon and one 24-hour inpatient visit.

Non-Limiting Exemplary Inclusion Criteria

Eligibility criteria include:
Healthy male or female
Age 18-65 years
BMI between 20 kg/m² and 35 kg/m² (inclusive)
Are willing to consume pre-prepared means
Have completed a high-dose (≥80 mIU/m²/min) euglycemic-hyperinsulinemic clamp during the last five years at Pennington Biomedical. The data from the previous PBRC clamp will be pulled and used in conjunction with the data from this study.
Willing to have blood and urine stored for future use

Non-Limiting Exemplary Exclusion Criteria

Participants will be ineligible to participate (or will be excluded from participating in this study) if they meet any of the following criteria:
Major lifestyle changes since the euglycemic-hyperinsulinemic clamp was performed (i.e. gain/lost weight, stopped smoking, began/stop exercise).
Unstable weight in the last 3 months [gain or loss>10 lb (or 4.5 kg)]
Diagnosed with diabetes
Untreated hypertension and average screening blood pressure>140/90 mmHg
Previous bariatric surgery (or other surgeries) for obesity or weight loss
Chronic use of medications affecting metabolism or sleep* *Sporadic use of these medications is fine (however, enrollment will depend on a case-by-case basis). If taking sporadically, participants are to not be taking the medication for 1-month prior to the first visit.*NOTE: Participants can also be excluded if deemed medically necessary by study Medical Investigator.
History of neurological disease
History of cardiovascular disease, or other chronic diseases, that can affect pancreatic or glucose metabolism.
Pregnant, planning to become pregnant, or breastfeeding
Adherence to special restrained diets (e.g., low CHO, low-fat, or vegetarian/vegan diets) over the last 3 months.

Non-Limiting Exemplary Recruitment

Twelve participants will be recruited, for example, aiming to enroll 6 men and 6 women. Participants will be recruited among those whose insulin sensitivity has been previously measured by a high-dose euglycemic-hyperinsulinemic clamp at during the last 5 years and indicated their wiliness to be recontacted for future research. Moreover, data from the euglycemic-hyperinsulinemic clamp will be requested and used in this study. Staff will explain the study and procedures to interested individuals and will provide them with a copy of the consent form for further explanation. Eligible individuals will be scheduled for a screening visit at PBRC.

Non-Limiting Exemplary Screening

Screening will take place during the first study visit (out of 3). Participants will provide informed consent prior to the initiation of study procedures. The informed consent process will be conducted primarily by the study coordinator, but also on occasion by the study investigators or by a trained clinic staff. Written consent will be obtained before any procedures are performed.

After providing informed consent, a medical history and a brief medical examination including measures of weight, height and blood pressure will be performed. If eligible, the participant body composition will be measure by a DXA scan and will be scheduled for the rest of study visits.

Non-Limiting Exemplary Study Visits

The study will consist of one screening visit, and two study visits taking place in consecutive days:

(a) Screening visit: Participants will arrive in the morning after an overnight fast (≥10 hours). The study will be described to participants and they will be offered to sign the consent form. If consented, the participants will have a medical history and a medical examination including measures of weight, height and blood pressure. If eligible, the participants' body composition will be assessed by a DXA scan.

(b) Visit 1: Visit 1 will be a 1-2 hour visit which will take place between 3 and 14 days after the screening visit. Upon arrival, the participants will be set up with a CGM. The participants will then be offered an individualized to-go dinner (25% estimated energy needs; 20% protein, 50% carbohydrates, 30% fat), and snack (10% estimated energy needs; 20% protein, 50% carbohydrates, 30% fat). Participants will be instructed to consume all food provided in the dinner and in the snack before 10 pm at the latest the day before visit 2.

c) Visit 2: Participants will arrive at about 7 am at an inpatient clinical unit, between 24 and 72 hours after visit 1, after an overnight fast, to spend 23 hours in the room calorimeter with discharge the next morning at around 7 am. All urines will be collected and pooled during the chamber stay, and a fasting blood draw will be taken immediately after leaving the chamber.

Non-Limiting Exemplary Measurements and Outcome Assessments

Body Composition (by Dual-Energy X-Ray Absorptiometry)

Without wishing to be bound by theory, a dual-energy X-ray absorptiometry (DXA) scan will be performed at the Imaging Facility at the screening visit. Total adiposity and regional fat mass will be assessed with DXA using a whole-body scanner (Lunar iDXA; General Electric, Milwaukee, Wis.). The DXA protocol requires that participants lie on a table wearing a hospital gown and with no metal objects on them while both legs will be placed together using two Velcro straps. The scanner emitting low energy X-rays and a detector passes along the body. The scan can take −10 minutes and the radiation dose is less than 1 mrem, equal to about 12-h of background radiation. The scans will be analyzed with the software version enCORE 13.6. We will run quality control scans on a daily basis, and GE has indicated that accuracy of the data is confirmed with these daily quality control scans. A pregnancy test will be performed before the scan on females of child-bearing potential. Fat mass will be calculated by taking the metabolic weight (i.e., total body weight minus gown weight) multiplied by the regional percent body fat given by the DXA.

Continuous Glucose Monitoring

Interstitial glucose will be assessed using CGM. Briefly, the abdominal area will be disinfected, and then trained staff from the Inpatient Unit will insert a glucose sensor under the skin in the abdominal area. The sensor has a small needle-like probe that inserts into the subcutaneous fat of the abdomen and that measures blood glucose levels without removing blood from the body. The sensor will then be attached to the recording unit, and the set-up will be secured with adhesive to the participant's body. The CGM device records blood sugar levels every 5 minutes. Two fingerstick will be done to measure glucose levels during the chamber stay (before breakfast on entering the chamber and before leaving the chamber the following morning), and used for CGM calibration.

Urinary C-Peptide and Nitrogen Excretion Rate.

Urine will be collected during the entire stay in the respiratory chamber. Urine volume and C-Peptide and nitrogen concentrations will be determined. Before entering the chamber, subjects will be required to void, and urines will be collected in a plastic container during the stay in the chamber. Moreover, urine will be archived.

Energy Expenditure and Macronutrient Oxidation Rate.

Total carbon dioxide production (VCO₂) and total oxygen consumption (VO₂) will be sampled and measured at 1-minute intervals over 23 hours in a respiratory chamber (whole-room calorimeter). This will be used to calculate respiratory quotient (RQ), energy expenditure (EE), and substrate oxidation rates. Subjects will enter the chamber at approximately 7:30 am and exit the following morning at 6.30 am. The chamber is a room about 12′×14′ with 2 windows, a bed, a desk and chair, a treadmill, a TV/VCR/DVD, a computer with internet access, a telephone, toilet facilities, motion sensors and a camera. Participants will be able to contact the nurse or chamber personnel by intercom, pager or phone at any time.

While in the chamber, participants will consume a eucaloric diet (50% carbohydrates, 30% fat and 20% protein). A standardized breakfast will be provided (approximately 8 am). Subjects will then be allowed approximately 4 hours of free time, after which lunch will be provided (approximately 12 pm). Lunch will be followed by approximately 6.5 hours of free time until dinner is served at approximately 7 pm. The 3 meals will be ingested over a maximum 30-minute period. Approximate energy balance will be maintained by the calculation of the average EE of the first 7 hours in the chamber and used to predict 24 hour EE using equations is Calories provided with the dinner meal will be adjusted so that energy intake equals predicted 24-hour EE, rounded to the nearest 100 Kcal.

During the 23-hour stay, subjects will not be allowed to sleep during the day, and the intensity of any physical activity can be no higher than walking around the chamber or stretching (no push-ups or sit-ups). After dinner, subjects will be given more free time until “lights out” at approximately 10:30 pm. Subjects will be awakened at approximately 06:30 am hour and instructed to void their bladder before leaving the chamber for CGM removal. Coffee and a granola bar will be offered upon discharge.

Non-limiting Example of Fasting Blood Draw

A fasting blood sample will be taken at visit 2, after leaving the metabolic chamber to assess glucose and insulin concentrations (for the determination of HOMA-IR). Moreover, blood will be archived.

Patient Safety and Confidentiality

Risks to Subjects: This Human Subjects Research meets the definition of a Clinical Trial. This study does not involve major risk to subjects. Efforts to minimize the potential risks of the assessment methods and outcome variables include frequent monitoring by the investigators to assure that no volunteer suffers any adverse effects from participating in the research.

The known risks, inconveniences, or side effects from the procedures in the project are included in Table 1.

Risks and Efforts to Minimize the Risks

| Procedure | Risks | Efforts to Minimize the Risk |
|---|---|---|
| Fasting for 10-h | Nausea | Light snacks will be |
| | | available to eat once fasting |
| | | procedures completed. |
| Continuous Glucose | Because CGM involves the | Aseptic (sterile) technique |
| Monitoring (CGM) | placement of an implantable | and trained personnel |
| | device below the skin, there | minimize these risks. |
| | is the possibility of | |
| | discomfort, pain, and | |
| | bruising at the site where the | |
| | device is inserted. There is | |
| | also a small risk of bleeding | |
| | and a very small risk of | |
| | infection at the site of the | |
| | blood draw. Finally, the | |
| | adhesive can cause redness | |
| | or irritation of the skin. | |
| Metabolic Chamber | Participants can experience | Participants can open the |
| | some level of claustrophobia | door of the chamber in |
| | or discomfort from staying in | case of an emergency. |
| | the chamber and being | Trained personnel also |
| | continuously monitored by a | minimize this risk. |
| | camera. The camera has been | |
| | installed for the participant's | |
| | safety and no one is allowed | |
| | access to the monitor except | |
| | chamber personnel. | |
| Blood Draw | Bruising, bleeding, pain, and | Trained phlebotomists and |
| | infection pose minimal risks. | personnel will use sterile |
| | | technique. |
| Dual-Energy X-Ray | DXA measures the amount | Exposure to X-rays is |
| Absorptiometry | of bone, muscle, and fat in | minimal. A pregnancy test |
| (DXA) | the body. The dose of | will be performed before the |
| | radiation is minimal, | DXA scans on females of |
| | equivalent to less than | child-bearing potential. |
| | 0.0004 mSv or ~12 h of | |
| | exposure to the sun. | |
| Confidentiality of | Taking part in this research | Study researchers and staff |
| Data | can involve providing | will take steps to protect data |
| | information that one | that is collected. Efforts, |
| | considers confidential or | such as coding research |
| | private. There is a slight risk | records, keeping research |
| | that data can be revealed | records secure and allowing |
| | inappropriately or | only authorized people to |
| | accidentally | access research records, will |
| | | be made to keep the data |
| | | safe. |

Safety Monitoring

In this study, an adverse event or experience is defined as any health-related unfavorable or unintended medical occurrence that happens throughout study participation. Examples of adverse events include but are not limited to the following:
A clinically significant laboratory or clinical test result.
An event that results in missing a study visit.
An event that requires a visit to a physician.
An event that occurs as a result of a study procedure.
Unanticipated or untoward medical events that can be study related.

We will use the provided definitions of Adverse Events and Serious Adverse Events. Events will be reported.

A serious adverse event (SAE) can refer to an unanticipated medical occurrence that is deemed associated with study participation by the study Medical Investigator that results in one of the following:
Death
Life-threatening event: Life threatening events in subjects can refer to those that in the view of the research staff and PI put the individual patient at imminent substantial risk of dying, or if continued participation in the study can result in death.
Hospitalization (initial or prolonged): Hospitalization or acute outpatient evaluation (e.g., in an emergency room) alone is not sufficient to qualify as a serious adverse event.
Any medical or surgical procedure performed (e.g., surgery that is not the planned oophorectomy, transfusion) itself is not the adverse event; instead, the condition that leads to the procedure is the adverse event.
Disability or permanent damage
If the adverse event resulted in a substantial disruption of the subject's ability to conduct normal life functions, i.e., the adverse event resulted in a significant, persistent or permanent change, impairment, damage or disruption in the patient's body function/structure, physical activities and/or quality of life.

Medical Intervention to prevent permanent impairment or damage

Surveillance and Reporting Procedures

All AEs from date of consent will be reported. Adverse events will be documented during the scheduled visits. For each sign, symptom or adverse event, the following information will be recorded:

A brief descriptor of the adverse event
Start and stop dates
Intensity (mild/moderate/severe)
Whether the AE was “serious” or not (as defined herein)
Causal association with the intervention assigned (none/doubtful/possibly/probably/very likely)
Outcome (resolved/resolved with sequelae/improving/still present and unchanged/death)
Action taken with respect to the intervention (none/intervention temporarily discontinued/medical therapy required/intervention permanently discontinued/other).

Adverse event data will be collected from the date of consent until the final visit. Without wishing to be bound by theory, adverse events to be absent or be mild and the subject will be able to resume activities within a day or two of reporting the event. Only adverse events that qualify as unanticipated problems will be reported. Unanticipated problems involving risks to subjects or others include incidents only if the incident is unexpected, related or possibly related to participation in the research, and indicated that subjects or others are at a greater risk of harm than was previously known or recognized. Any action resulting in a temporary or permanent suspension of this study (e.g., IRB actions, or actions by the PI and/or co-investigators) will be reported to the appropriate officials.

Withdrawal of Subjects

There is no risk associated with withdraw from the study Participants can be withdrawn from the study if the PI or MI feels that their continued participation can jeopardize the participant's health or the results of the study.

Stopping Rules

This study does not involve major risk for participating. Nevertheless, in addition to monitoring recruitment, we also will monitor the rates of adverse events in our subjects. The study investigators will alert the IRB, if a larger than expected adverse event rate occurs in our subjects.

ClinicalTrails.gov Requirements

The clinical study will be registered on ClinicalTrials.gov, in accordance with NIH recommendations. The unique NCT identifier will be included in all future Progress Reports and publications.

Data Management

Sample Size Considerations

Participants with different levels of IS as assessed by the hyperinsulinemic-euglycemic clamp will be recruited. Thus, notable inter-individual variability will be observed in the 24 urinary C-peptide/24 h serum glucose levels ratio. Without wishing to be bound by theory, the trial will be used to generate first-of-its-kind data for our investigative team to validate effect sizes for a future study.

Statistical Analyses

A statistician will perform linear regression analyses to assess the association between the 24 urinary C-peptide/24 h serum glucose levels ratio and the M-value from the hyperinsulinemic-euglycemic clamp. Moreover, models will be used to analyze the association between the 24 urinary C-peptide/energy intake ratio and the M-value from the hyperinsulinemic-euglycemic clamp.

### REFERENCES CITED IN THIS EXAMPLE

1. Artunc, F. et al. The impact of insulin resistance on the kidney and vasculature. Nature Reviews Nephrology 12, 721-737 (2016).
2. Saklayen, M. G. The Global Epidemic of the Metabolic Syndrome. Current Hypertension Reports 20, (2018).
3. Baum, A. et al. Targeting weight loss interventions to reduce cardiovascular complications of type 2 diabetes: a machine learning-based post-hoc analysis of heterogeneous treatment effects in the Look AHEAD trial. Lancet Diabetes Endocrinol. 5, 808-815 (2017).
4. Ling, C. K. et al. Insulin resistance and inflammation predict kinetic body weight changes in response to dietary weight loss and maintenance in overweight and obese subjects by using a Bayesian network approach1-4. Am. J. Clin. Nutr. 98, 1385-1394 (2013).
5. Pennings, N., Jaber, J. & Ahiawodzi, P. Ten-year weight gain is associated with elevated fasting insulin levels and precedes glucose elevation. Diabetes. Metab. Res. Rev. 34, e2986 (2018).
6. Morrison, J. A., Glueck, C. J., Horn, P. S., Schreiber, G. B. & Wang, P. Pre-teen insulin resistance predicts weight gain, impaired fasting glucose, and type 2 diabetes at age 18-19 y: a 10-y prospective study of black and white girls. Am. J. Clin. Nutr. 88, 778-788 (2008).
7. Petersen, M. et al. Randomized, multi-center trial of two hypo-energetic diets in obese subjects: High- versus low-fat content. Int. J. Obes. 30, 552-560 (2006).
8. Mediano, M. F. F. & Sichieri, R. Insulin Resistance Predicts the Effectiveness of Different Glycemic Index Diets on Weight Loss in Non-Obese Women. Obes. Facts 5, 641-647 (2012).
9. Cummings, D. M., Henes, S., Kolasa, K. M., Olsson, J. & Collier, D. Insulin resistance status: Predicting weight response in overweight children. Arch. Pediatr. Adolesc. Med. 162, 764-768 (2008).
10. Hjorth, M. F. et al. Personalized nutrition: pretreatment glucose metabolism determines individual long-term weight loss responsiveness in individuals with obesity on lowcarbohydrate versus low-fat diet. Int. J. Obes. 43, 2037-2044 (2019).
11. Galgani, J. E. et al. Urinary C-peptide excretion: A novel alternate measure of insulin sensitivity in physiological conditions. Obesity 18, 1852-1857 (2010).
12. Venkatesan, P. et al. Surrogate measures of insulin sensitivity when compared to euglycemic hyperinsulinemic clamp studies in Asian Indian men without diabetes. J. Diabetes Complications 30, 287-291 (2016).
13. Ciresi, A., Guarnotta, V., Pizzolanti, G. & Giordano, C. Comparison between euglycemic hyperinsulinemic clamp and surrogate indices of insulin sensitivity in children with growth hormone deficiency. Growth Horm. IGF Res. 39, 40-44 (2018).
14. Klonoff, D. C., Ahn, D. & Drincic, A. Continuous glucose monitoring: A review of the technology and clinical use. Diabetes Research and Clinical Practice 133, 178-192 (2017).
15. Lam, Y. Y. et al. Determinants of sedentary 24-h energy expenditure: equations for energy prescription and adjustment in a respiratory chamber. Am. J. Clin. Nutr. 99, 834-42 (2014).

### Example 3—CGM-PEPTIDE

Development of a Method to Measure Insulin Sensitivity in Humans

Background—Insulin Sensitivity
Insulin resistance, the lack of insulin sensitivity, is a risk factor for multiple pathological conditions, such as type 2 diabetes mellitus and cardiovascular disease
The euglycemic-hyperinsulinemic clamp is the standard for the assessment of insulin sensitivity in humans
Unfortunately, the clamp method is only available in a certain laboratories

Background—Euglycemic-hyperinsulinemic clamp
However, this method is expensive, presents high burden for participants and assess insulin sensitivity under non-physiological conditions

Background—C-Peptide
C-peptide is secreted with insulin and its urinary excretion rate provides an indirect, non-invasive marker of insulin secretion
The 24-hour C-peptide urinary excretion rate, adjusted for energy intake, is better correlated with insulin sensitivity than other indirect methods. (Galgani et al; Obesity 18, 1852-57, 2010) (FIG. 4).
However, the circulating glucose concentration is the major stimulus for insulin secretion and it varies in response to identical food intake from one person to another.

CGM-Peptide Study

Without wishing to be bound by theory, adjusting C-peptide urinary excretion rate for average glucose concentration instead of energy/nutrient intake can improve the prognosis of insulin sensitivity.

Without wishing to be bound by theory, the study will determine whether the ratio between 24 h C-peptide urinary excretion rate and the average 24 h blood glucose represents a valid and reliable measure of insulin sensitivity when compared to the rate of glucose disposal during a euglycemic-hyperinsulinemic clamp

CGM-Peptide Study—Non-Limiting Exemplary Methods

12 participants (18-65 y, 20-35 kg/m²) will have their insulin sensitivity assessed by a 2-step euglycemic-hyperinsulinemic clamp.

They will spend 24 hours domiciled in a metabolic chamber, wearing a continuous glucose monitoring and collecting a 24-h urine sample, to assess urinary C-Peptide excretion rate. Body composition and fasting glucose and insulin will also be assessed.

The association between the ratio of average 24-h blood glucose/24-h urinary C-peptide and the glucose disposal rate from a euglycemic-hyperinsulinemic clamp will be analyzed and compared to other insulin sensitivity markers.

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

What is claimed:

1. A method of identifying a subject with insulin resistance, the method comprising:
determining the subject's blood glucose concentration,
determining the subject's blood or urinary C-peptide level
calculating the ratio of the subject's blood glucose concentration to the blood or urinary C-peptide concentration,
identifying a subject with insulin resistance if this ratio is high; or
identifying a subject with normal insulin sensitivity if this ratio is low; and
treating the subject.

2. The method of claim 1, wherein treating the subject comprises administering to the subject one or more insulin sensitizers.

3. The method of claim 1, wherein blood glucose concentration is measured continuously or periodically.

4. The method of claim 1, wherein blood glucose concentration is measured over a period of time.

5. The method of claim 5, wherein the period of time comprises about 24 hours.

6. The method of claim 1 wherein blood or urinary C-peptide level is measured over a period of time.

7. The method of claim 6, wherein the period of time comprises about 24 hours.

8. The method of claim 1, wherein the blood glucose concentration and the urinary C-peptide level are measured simultaneously or sequentially.

9. The method of claim 1, wherein the subject's blood glucose concentration is measured using a transcutaneous glucose monitor (CGM).

10. The method of claim 1, wherein the subject's blood or urinary C-peptide level is measured using a transcutaneous monitor or a dipstick.

11. A method of identifying a subject with insulin resistance, the method comprising:
determining the subject's blood or urinary C-peptide level,
normalizing the subject's blood or urinary C-peptide level for the subject's blood glucose concentration,
identifying the subject as having insulin resistance if the subject's normalized blood or urinary C-peptide level is above a threshold, and
treating the subject.

12. The method of claim 11, wherein the subject is identified as not having insulin resistance if the normalized urinary C-peptide level is below a threshold.

13. The method of claim 11, wherein treating the subject comprises administering to the subject one or more insulin sensitizers.

14. The method of claim 11, wherein blood glucose concentration is measured continuously or periodically.

15. The method of claim 11, wherein blood glucose concentration is measured over a period of time.

16. The method of claim 15, wherein the period of time comprises about 24 hours.

17. The method of claim 11, wherein blood or urinary C-peptide level is measured over a period of time.

18. The method of claim 17, wherein the period of time comprises about 24 hours.

19. The method of claim 11, wherein the blood glucose concentration and the blood or urinary C-peptide level are measured simultaneously or sequentially.

20. The method of claim 11, wherein the subject's blood glucose concentration is measured using a transcutaneous glucose monitor (CGM).

21. The method of claim 11, wherein the subject's blood or urinary C-peptide level is measured using a transcutaneous monitor or a dipstick.

22. A method of identifying a subject with or at risk of a disease characterized by insulin resistance, the method comprising:
determining the subject's blood glucose concentration,
determining the subject's blood or urinary C-peptide level,
calculating a ratio of the subject's blood glucose concentration to the subject's blood or urinary C-peptide level, thereby identifying a subject with or at risk of a disease characterized by insulin resistance if the ratio is high, and
treating the subject.

23. The method of claim 22, wherein the disease comprises diabetes or cardiovascular disease.

24. The method of claim 23, wherein diabetes comprises type 2 diabetes mellitus.

25. The method of claim 22, wherein the subject is identified as not having or not at risk of a disease characterized by insulin resistance if the ratio is low.

26. The method of claim 22, wherein treating the subject comprises administering to the subject one or more insulin sensitizers.

27. The method of claim 22, wherein blood glucose concentration is measured continuously or periodically.

28. The method of claim 22, wherein blood glucose concentration is measured over a period of time.

29. The method of claim 28, wherein the period of time comprises about 24 hours.

30. The method of claim 22, wherein blood or urinary C-peptide level is measured over a period of time.

31. The method of claim 30, wherein the period of time comprises about 24 hours.

32. The method of claim 22, wherein the blood glucose concentration and the blood or urinary C-peptide level are measured simultaneously or sequentially.

33. The method of claim 22, wherein the subject's blood glucose concentration is measured using a transcutaneous glucose monitor (CGM).

34. The method of claim 22, wherein the subject's blood or urinary C-peptide level is measured using a transcutaneous monitor or a dipstick.

35. A method of identifying a subject with or at risk of a disease characterized by insulin resistance, the method comprising:
determining the subject's blood or urinary C-peptide level,
normalizing the subject's blood or urinary C-peptide level for the subject's blood glucose concentration,
identifying the subject as having insulin resistance if the subject's normalized blood or urinary C-peptide level is above a threshold, and
treating the subject.

36. The method of claim 35, wherein the disease comprises diabetes or cardiovascular disease.

37. The method of claim 36, wherein diabetes comprises type 2 diabetes mellitus.

38. The method of claim 35, wherein the subject is identified as not having or not at risk of a disease characterized by insulin resistance if the normalized blood or urinary C-peptide level is below a threshold.

39. The method of claim 35, wherein treating the subject comprises administering to the subject one or more insulin sensitizers.

40. The method of claim 35, wherein blood glucose concentration is measured continuously or periodically.

41. The method of claim 35, wherein blood glucose concentration is measured over a period of time.

42. The method of claim 41, wherein the period of time comprises about 24 hours.

43. The method of claim 35, wherein blood or urinary C-peptide level is measured over a period of time.

44. The method of claim 43, wherein the period of time comprises about 24 hours.

45. The method of claim 35, wherein the blood glucose concentration and the blood or urinary C-peptide level are measured simultaneously or sequentially.

46. The method of claim 35, wherein the subject's blood glucose concentration is measured using a transcutaneous glucose monitor (CGM).

47. The method of claim 35, wherein the subject's blood or urinary C-peptide level is measured using a transcutaneous monitor or a dipstick.

48. A method of determining a subject's C-peptide level, the method comprising
obtaining a blood and/or urine sample from a subject,
measuring the blood or urinary C-peptide level,
measuring blood glucose concentration in the subject, and
normalizing the blood or urinary C-peptide level for the subject's blood glucose concentration, and thereby determining the subject's C-peptide level.

49. The method of claim 48, wherein the subject's C-peptide level is an indicator of the subject's resistance to insulin.

50. The method of claim 48, wherein the subject's C-peptide level is an indicator of the subject having or at risk of having a disease characterized by insulin resistance.

51. The method of claim 50, wherein the disease comprises diabetes or cardiovascular disease.

52. The method of claim 51, wherein diabetes comprises type 2 diabetes mellitus.

53. A kit, wherein the kit comprises one or more components and reagents to measure
blood glucose concentration in a subject,
blood C-peptide level in a subject, or
urinary C-peptide in a subject.
